(19) United States
(12) Patent Application Publication
(10) Pub. No.: US 2021/0282716 A1
(43) Pub. Date: Sep. 16, 2021

(54) Awake-Animal Imaging Restrainer System for Animal Models of Brain Diseases

(71) Applicant: Samuel R. Cramer, Georgetown, SC (US)

(72) Inventor: Samuel R. Cramer, Georgetown, SC (US)

(21) Appl. No.: 16/929,490

(22) Filed: Jul. 15, 2020

Related U.S. Application Data
(60) Provisional application No. 62/875,467, filed on Jul. 17, 2019.

(51) Int. Cl. A61B 5/00; A61B 5/055; A61D 3/00; A61D 7/04

(52) U.S. Cl. CPC A61B 5/702; A61B 5/055; A61D 3/00; A61D 2003/006; A61B 2503/40; A61B 2503/42; A61D 2003/003; A61D 7/04

(57) ABSTRACT

A restrainer system for performing awake-animal imaging on a rodent. The system comprises a front section, a first pair of adjustable side pieces for contacting with a snout of the rodent from both sides, a second pair of adjustable side pieces for contacting with a rear jaw of the rodent from both sides, a front section, first floating component for applying pressure to a nose section of the rodent during testing and a mouthpiece for holding the rodent's teeth. That front section connects to a middle section having a floor element upon which to rest a main body of the rodent and all four limbs, a pair of adjustable walls for applying pressure to the rodent's back legs, and a middle section floating component for applying pressure to the rodent's shoulder blades from above. The latter connects to a rear section having a plateau onto which to rest the rodent's tail, and a rear section floating component for securing over the rodent's tail. A method of use is also disclosed.

### CROSS-REFERENCE TO RELATED APPLICATIONS

This application is a perfection of U.S. Provisional Ser. No. 62/875,467, filed on Jul. 17, 2019, the disclosure of which is fully incorporated by reference herein.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB).

Not Applicable.

### STATEMENT REGARDING PRIOR DISCLOSURES BY THE INVENTOR OR A JOINT INVENTOR

Not Applicable.

### BACKGROUND OF THE INVENTION

### (1) Field of the Invention

These inventions relate to testing equipment for the awake-animal imaging of laboratory test rats. When reconfigured, mostly for size/scale, they could also work for use in the testing of laboratory mice. These inventions are all intended to minimize rodent subject head movement during the acquisition of MRI and fMRI data. The system should improve the quality and fidelity of data obtained from such test scans when rodent subjects are fitted with these devices. The inventions particularly relate to restrainer systems for rats/mice awake-imaging and further include an added emphasis to animal well being following data acquisition (i.e. these improvements minimize bodily injury to the animal); and further to their methods of use.

### (2) Description of Related Art including Information Disclosed Under 37 CFR 1.97 and 1.98

Previous solution and issues: Most labs utilize an equipment setup outlined in King et al.'s 2005 paper on awake-animal imaging.¹ Briefly, that equipment involves a long cylindrical tube where the rat is placed within longitudinally. The forearms are typically taped together as are the back legs. These bindings and the physical boundary of the cylindrical tube serve as the primary means of minimizing body movement and ultimately head movement during imaging procedures. Unfortunately, this method often leads to inflammation and potentially structural damage to the front paws. Moreover, the tape can cut off circulation to the appendages making the setup uncomfortable for the rat. And, animal respirations can be compromised due to the abnormal body postures the animals are forced to occupy which is a consequence of these more traditional setup design choices. These instances ultimately lead to pain and stress that can be deleterious to the quality of awake-animal imaging studies: in particular, those that subsequently involve animal behavior. ¹King, Jean A. et al. “Procedure for minimizing stress for fMRI studies in conscious rats.” Journal of Neuroscielice Methods, vol 148, no. 2, 2005, pp 154-160, doi: 10 1016/j.jneumeth.2005.04.011.

Also of importance, most of the devices currently used by labs lack an ergonomically friendly shape that does not match the anatomy of the rat. For example, the simple body tube outlined by King et al. is a cylinder. However, preferred resting body postures for rats and mice are not compatible with this cylindrical shape. A primary purpose behind my proposed design is to minimize the risk of injury to the rat while minimizing overall rodent head motion during the imaging acquisition. Further, the restrainer provides an environment that promotes stressor contextualization, which reduces long-term effects of imaging-related stress. This is important for studies that subsequently acquire behavioral data after the imaging has completed.

Patents relevant to the general field of body tubes and their use in animal testing include, in chronological order: Ferris et al U.S. Pat. No. 6,711,430; Ferris et al U.S. Pat. No. 6,873,156 and Geddes et al U.S. Pat. No. 7,096,059.

### BRIEF SUMMARY OF THE INVENTION

There are multiple product considerations here including: a rodent, especially a rat or mouse head restraint system including a rodent head/neck holder for laboratory testing; a holder for the rodent's upper body; a lower body and leg restraint; and a tail restraint for the same. The design of this invention allows for comfortable positioning of the animals' forearms, allowing them a greater degree of positional flexibility and freedom. Yet another disclosure herein addresses a method for using all of the aforementioned devices preferably for use with laboratory rats but re-sized and/or slightly reconfigured for use with smaller mice.

One preferred embodiment of this invention addresses a restrainer system for performing awake-animal imaging on a rodent. That restrainer system comprises a front section having, at least: a floor element, a first pair of adjustable side pieces for contacting with a snout of the rodent from both sides, a second pair of adjustable side pieces for contacting with a rear jaw of the rodent from both sides, a front section, first floating component for applying pressure to a nose section of the rodent during testing and a mouthpiece on which the rodent's head rests. Applying pressure to the nose section ensures that the rodent's teeth remain engaged with the “mouthpiece” component described later herein.

A second pair of adjustable side pieces (Elements 10A and 10B) elevate the rodent's head when an Element 7 (below) is present for interfacing with an experimenter's head post implantation or “headcap” when using such a device. When an Element 7 is not being used, Elements 10A and 10B serve to restrict lateral motion of the rodent's head.

The system further requires a middle section having, at least: a floor element upon which to rest a main body of the rodent and all four limbs (i.e., the forearms and the rear legs), a pair of adjustable walls for applying pressure to the rodent's back legs, and a middle section floating component for applying pressure to the rodent's shoulder blades from above. Lastly, there is a rear section having, at least: a plateau (or mostly flat planar) section (or “shelf”) onto which to rest the rodent's tail, and a rear section floating component for securing over the rodent's tail. This “plateau”, at the very end of the rear piece, is used to facilitate user-rotation of the overall restrainer system when it is loaded into the bore of an MRI scanner.

A method of using the aforementioned assemblies is also disclosed.

### BRIEF DESCRIPTION OF THE DRAWINGS

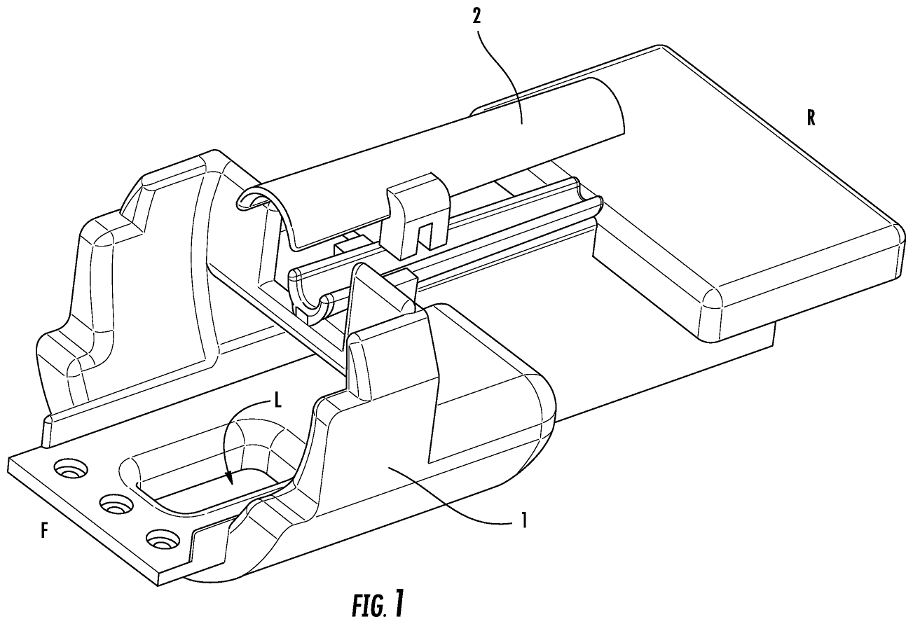

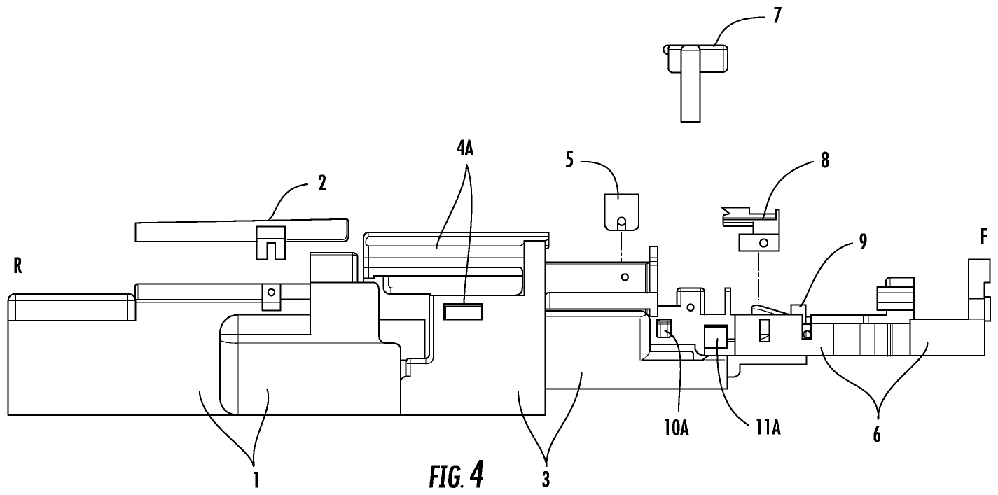

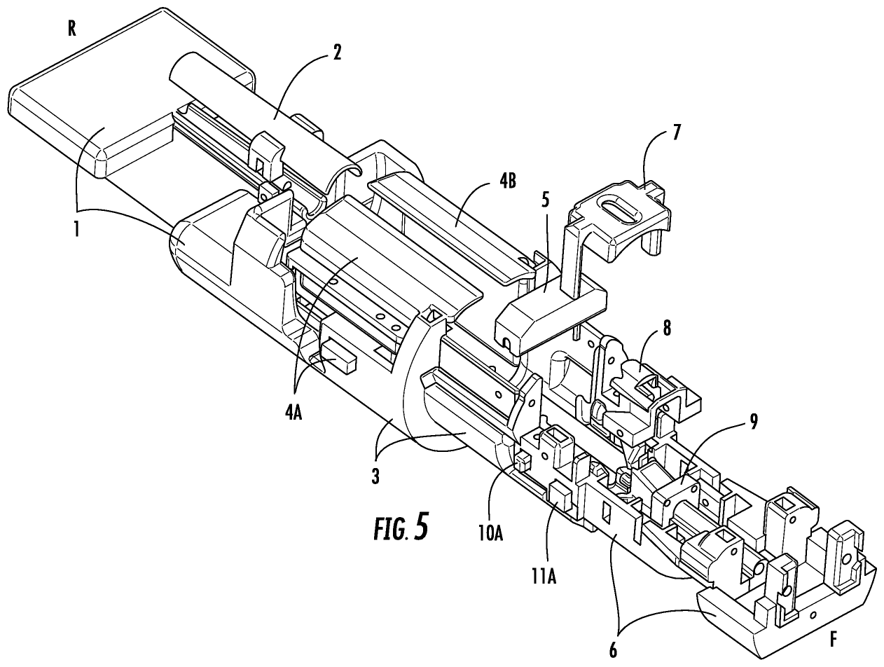

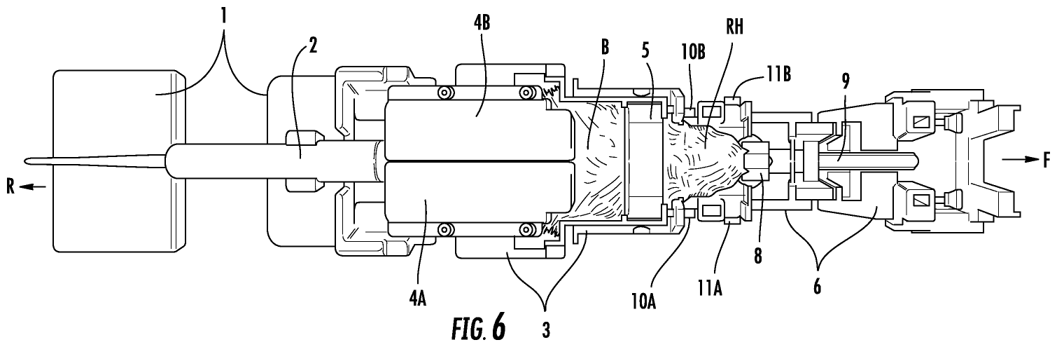

Further features, objectives and advantages of this invention will be made clearer with respect to the following detailed description of preferred embodiments made with reference to the accompanying drawings. They are broken into three main groups, the first being component parts (FIGS. 1 through 3), the fully assembled unit (FIGS. 4 and 5), then one representative view showing a rat positioned in the unit with its head toward the top (FIG. 6).

### DETAILED DESCRIPTION OF THE INVENTION

The items in the first set are commonly color-coded to show how they fit together in the second set.

In these accompanying views:

FIG. 1 is a right side, upper perspective view of the rear (or back) third of this invention, i.e., the section responsible for housing the rodent's tail on a shelf or “plateau”, and a portion for holding the rodent's posterior. The latter includes a latrine section L for housing urine and feces. FIG. 1 also shows a floating piece (shown suspended above) for holding the animal's upper tail in place;

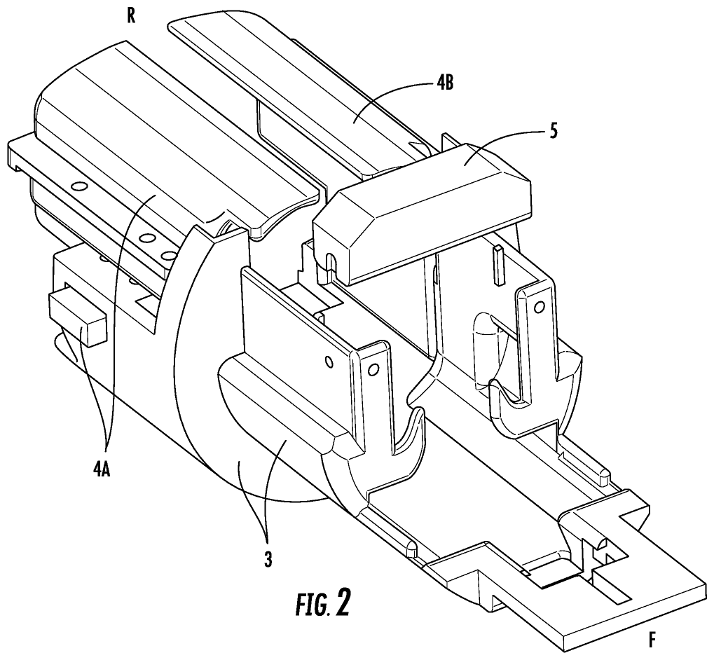

FIG. 2 is a left upper front perspective view of the middle third of Applicant's assembly; it is used to hold (or house) the rodent's main body and all four limbs (2 legs and 2 forearms). It likewise includes a floating piece towards of the “front end” of this middle section designed for lowering to apply pressure on the rodent's shoulder blades. Also included in this middle third section are pieces (Elements 4A and 4B below) that apply pressure to the rodent's hind legs. Element 4 can be broken down into two subcomponents: (i) a wall portion that applies pressure to the sides of the rodent's legs and minimizes lateral lower body motion; and (ii) a “ceiling” that prevents the rodent from jumping up-and-down (i.e. limits dynamic vertical movement).

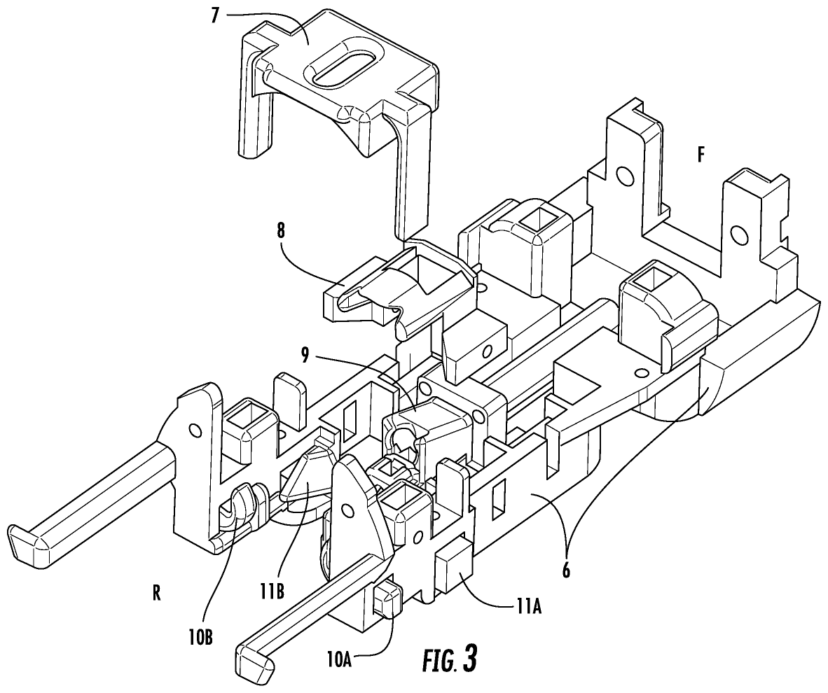

FIG. 3 is a left upper perspective view of the forward most, or front third of Applicant's restrainer system assembly for housing the rodent's head during laboratory testing. It has a mouthpiece component into which the rodent's teeth are inserted, along with four adjustable side-pieces for contacting the rodent's snout and rear jaw from both (left and right) sides. A first floating piece to this subcomponent is lowered into the assembly for applying pressure to the rodent's nose; and an optional, second floating piece is designed for integrating with surgically implanted headcaps, when practitioners elect to use the same;

FIG. 4 is an exploded, left side sectional view of the entire assembly, with the middle section joined at the ends to: the rear third section of FIG. 1 on the left side of FIG. 4; and to the front subcomponent section from FIG. 3, towards the right side of FIG. 4 as shown;

FIG. 5 is an upper perspective view from the left front side of the whole assembly shown in FIG. 4. This view includes several “floating” pieces shown hovering over where they will be installed (See especially, the descriptions for Elements 2, 5, 7 and 8 below).

Lastly, FIG. 6 is a top plan view of the whole assembly that has a representative rat (rodent) positioned for securing therein, said rat's body being visible through portions of the device's middle section and the rat's head being held in place toward the right side of the same device as shown in this FIG. 6. Note that Element 7 (below) has been omitted from this view to better understand the placement/positioning of the rat's head in relation to the other component pieces of this invention.

In the accompanying FIGURES, various subcomponents to the rear, middle and front units (FIGS. 1, 2 and 3, respectively) are commonly numbered even though they may appear in different viewpoints and from different (left or right, top or side) perspectives. It is to be understood that in these views, directional indicators are added for designating the front F and rear R “ends” of the assembly itself and the three main “thirds” making up this configuration. The respective rear, middle and front thirds are intended to be connected together with 8×32 screws (not shown), or their equivalents. Still other connectors may suffice.

The drawing key for the respective subcomponent parts is as follows:

Element 1 is a secondary feature (i.e., not a primary CORE feature) and comprises a spaced apart tail holding plateau element which may be divided into a more forward and a more rearward sub-half In the accompanying FIGS., Element 1 is indicated with either a single arrow or two arrows to the same part.

Element 2 is the upper portion to the floating tail cover that joins to a mated subpart of Element 1 after the rodent's tail is positioned between. Like Element 1, Element 2 is not a primary CORE feature of the invention—but more secondary or “optional”). By optional, it is meant that the component, as depicted, may be replaced by a simpler design or style. Nevertheless, the restrainer system of this invention needs a back or rear third that connects to the middle third. All other features that follow ARE primary core features and critical to the proper positioning of a rodent in said device for improved restraint during laboratory testing, especially the performance of waking MRI and/or fMRI scans on rodents/subjects “held” therein.

Element 3 is the multi-sectional, middle component rodent floor assemblies (shown by a pair of arrows to the rearward most part and a more forward, smaller dimensional section). When a rodent is first set up in this restrainer, prior to imaging, sedation is required. Element 9 below has a cylindrical conduit that allows for easy administration of sedating agents for set up purposes. In the context of the whole assembly, a rodent's main body (RB in FIG. 6) is carefully laid onto these floor assemblies, its head (H) towards the front of the device before two 2 piece components (Elements 4A and 4B, respectively), each of them comprised of a vertical wall and an overhanging curved roof, are positioned over the rodent's posterior and secured to the floor assemblies (Element 3). In the accompanying FIGS., Element 4A is indicated with either a single arrow or two arrows to the same part.

Elements 4A and 4B are comprised of a wall portion and a “ceiling” portion with the wall portion minimizing lateral lower body motion and the “ceiling” portion minimizing vertical lower body motion by the rodent.

Element 5 is a floating “piece” for the middle, or intermediate, section of this restraint assembly. It is intended for lowering in place to apply pressure on the rodent's shoulder blades. That applied pressure minimizes upper body movement of the rodent within the device while helping to stabilize a rearmost portion of the rodent's skull where its cerebellar and brainstem nuclei are located.

Element 6 (shown in the accompanying FIGS. using two arrows) is the floor element for the forward most “third” of this apparatus/device. Element 6 consists of four adjustable side pieces for contacting the rodent from both (left and right) sides of its rear jaw (Elements 10A and 10B) and snout (Elements 11A and 11B). This component includes easily accessible screws that can be rotated for adjusting the positioning of the MM coil to accommodate different rodent skull heights.

Element 7 is one of the first floating pieces to this front (head holding) section. It is intended for lowering over the rodent's skull and interfacing with head-post implants (or “headcaps”) when an experimenter chooses to incorporate the same into his/her experiments. Element 8 is the other floating piece to the front “third” section of this invention. It is used to apply pressure to the rodent's nose and keep its teeth engaged with Element 9.

Element 9 is used for “housing” the teeth of the rodent. It also provides a cylindrical conduit for anesthesia delivery.

Elements 10A and 10B are the left and right adjustable side pieces for contacting the rodent's rear jaw. Depending on whether an experimenter uses an Element 7 (above), the functionality of these pieces change. If a head post implant is present, Elements 10A and 10B are used to elevate the head into appropriate position. If the head post implant is not used, Elements 10A and 10B simply minimize lateral movement. By further explanation, a “head post implant” is a surgical procedure that involves (in brief) gluing a purposefully designed piece of plastic onto the skull of the animal. This implant can subsequently interface with a surrounding stable structure (in our case, the restrainer), and greatly minimize head motion.

Elements 11A and 11B are the left and right adjustable side pieces for contacting the rodent's snout.

### EXAMPLE

### Head Holding Subassembly (or Front Third)

This head subassembly houses the head of the rodent. It is where the MRI/fMRI coil rests during data acquisition. This piece interfaces with the body component, mastoid contacts (Elements 10A and 10B above), zygomatic contacts (i.e., Elements 11A and 11B), mouthpiece, nose block, and screws that function as coil elevators.

### Body Subassembly (or Middle Third)

The body subassembly houses the rodent's torso, forearms and rear legs. This piece interfaces with the scapula contact (Element 5 above), leg subassembly, head subassembly and Element 4 above.

### Tail Subassembly (or Back/Rear Third)

The last subassembly unit houses the rodent's tail on a plateau and a portion of the rodent's posterior. This subassembly is meant to connect to, and interface with, the middle (intermediate or body) subassembly.

### Mouthpiece

The mouthpiece, Element 9 above, stabilizes the head of the rodent by securing the rat's upper teeth. A cylindrical conduit at its front permits the delivery of vapor. This piece interfaces with the head component where it is screwed in.

### Nose Block

The nose block (Element 8) stabilizes the head of the rodent by applying a downward pressure along the snout of the rat. This downward pressure ensures that the rat cannot lift its head out of the mouthpiece. This piece interfaces with the head subassembly (Element 6) where it is screwed in.

### Mastoid Contacts

The mastoid contacts, of which there are two (Elements 10A and 10B), apply pressure to the left and right mastoid process of the rodent directly behind its ear. This minimizes lateral motion of the rodent's head during imaging. These pieces interface with the head subassembly where they are screwed in.

### Zygomatic Contact

The zygomatic contacts, of which there are two (Elements 11A and 11B), apply pressure to the left and right zygomatic process of the rodent below its eye around the transition area to the maxilla portion of the face. This minimizes lateral motion of the rodent's head during imaging. These pieces interface with the head subassembly where they are screwed in.

### Scapula Contact

The scapula contact (Element 5) applies downwards pressure on to the scapula of the rodent. That pressure minimizes movement of the animal's chest and upper back while also ensuring that its forearms remain secured in the body component. This piece interfaces with the body subassembly where it is screwed in.

### Upper Tail Attachment

The upper tail attachment (Element 2) is the ceiling to the lower tail attachment and minimizes the rat's tail motion during imaging. This piece interfaces with the lower tail attachment where it is screwed in.

### SEQUENCE LISTING

Not Applicable

Having described the best modes currently known for practicing this axle replacement system and method, it is to be understood that the scope of this invention may be further described by the attached claims.

What is claimed is:

1. A restrainer system for performing awake-imaging on a rodent, said restrainer system comprising:
(a) a front section having, at least:
(i) a floor element,
(ii) a first pair of adjustable side pieces for contacting with a snout of the rodent from both sides,
(iii) a second pair of adjustable side pieces for contacting with a rear jaw of the rodent from both sides,
(iv) a front section, first floating component for applying pressure to a nose section of the rodent during testing; and
(v) a mouthpiece component on which to rest the rodent's head and houses the rodent's teeth;
(b) a middle section having, at least:
(i) a floor element upon which to rest a main body of the rodent and all four limbs,
(ii) a pair of adjustable walls for applying pressure to the rodent's back legs, and
(iii) a middle section floating component for applying pressure to the rodent's shoulder blades from above; and
(c) a rear section having, at least:
(i) a plateau section onto which to rest the rodent's tail, and
(ii) a rear section floating component for securing over the rodent's tail.

2. The restrainer system of claim 1 wherein the mouthpiece component of the front section includes a conduit for anesthesia delivery to the rodent.

3. The restrainer system of claim 1 wherein the rodent is a rat.

4. The restrainer system of claim 1 wherein the rodent is a mouse.

5. The restrainer system of claim 1 wherein the front section further comprises
(vi) a front section, second floating component for integrating with surgically implanted headcaps.

6. A restrainer system for humanely performing experimentation on a laboratory rat, said restrainer system comprising:
(a) a front section having, at least: a floor element, a first pair of adjustable side pieces for contacting with a snout of the laboratory rat from both sides, a second pair of adjustable side pieces for contacting with a rear jaw of the laboratory rat from both sides, a front section, first floating component for applying pressure to a nose section of the laboratory rat during testing, a mouthpiece component for housing the laboratory rat's teeth and a front section, second floating component for integrating with surgically implanted headcaps when used;
(b) a middle section having, at least: a floor element upon which to rest a main body of the laboratory rat and all four limbs, a pair of adjustable walls for applying pressure to the laboratory rat's back legs, and a middle section floating component for applying pressure to the laboratory rat's shoulder blades from above; and
(c) a rear section having, at least: a plateau section onto which to rest the laboratory rat's tail, and a rear section floating component for securing over the laboratory rat's tail.

7. The restrainer system of claim 6 wherein the mouthpiece component includes a conduit for anesthesia delivery to the laboratory rat.

8. The restrainer system of claim 6 wherein the experimentation includes performing awake-animal imaging on the laboratory rat.

9. The restrainer system of claim 6 wherein the experimentation includes acquiring MRI or fMRI data from the laboratory rat.

10. A method for restraining a rodent for performing experimental testing thereon, said method comprising the steps of:
(a) providing a restrainer system comprising:
(i) a front section having, at least: a floor element upon which to rest a head of the rodent, a first pair of adjustable side pieces for contacting with a snout of the rodent from both sides, a second pair of adjustable side pieces for contacting with a rear jaw of the rodent from both sides, a front section, first floating component for applying pressure to a nose section of the rodent during testing, and a mouthpiece component for housing the rodent's teeth;
(ii) a middle section having, at least: a floor element upon which to rest a main body of the rodent and all four limbs, a pair of adjustable walls for applying pressure to the rodent's back legs, and a middle section floating component for applying pressure to the rodent's shoulder blades from above; and
(iii) a rear section having, at least: a plateau section onto which to rest the rodent's tail, and a rear section floating component for securing over the rodent's tail; and
(b) positioning the rodent within the front section, middle section and rear section of the restrainer system.

11. The method of claim 11 wherein the mouthpiece in the front section includes a conduit for anesthesia delivery to the rodent.

12. The method of claim 11 wherein the front section further comprises a front section, second floating component for integrating with surgically implanted headcaps when used.

13. The method of claim 11 wherein the rodent is a rat.

14. The method of claim 11 wherein the rodent is a mouse.

15. The method of claim 11 wherein the front section further comprises a front section, second floating component for integrating with surgically implanted headcaps.
